(19) United States
(12) Patent Application Publication
(10) Pub. No.: US 2026/0128179 A1
(43) Pub. Date: May 7, 2026

(54) STATE TRANSITION ESTIMATION DEVICE, STATE TRANSITION ESTIMATION METHOD, AND NON-TRANSITORY COMPUTER-READABLE MEDIUM

(71) Applicant: NEC CORPORATION, Tokyo (JP)

(72) Inventors: Kosuke NISHIHARA, Tokyo (JP); Fumiyuki Nihey, Tokyo (JP); Keisuke Suzuki, Tokyo (JP); Mana Hashimoto, Tokyo (JP); Yuki Kosaka, Tokyo (JP); Kentaro Nakahara, Tokyo (JP)

(73) Assignee: NEC CORPORATION, Tokyo (JP)

(21) Appl. No.: 19/358,557

(22) Filed: Oct. 15, 2025

(30) Foreign Application Priority Data
Nov. 6, 2024 (JP) 2024-194452

(51) Int. Cl. G16H 50/70; G06T 11/20; G16H 50/30

(52) U.S. Cl. CPC G16H 50/70; G06T 11/26; G16H 50/30

(57) ABSTRACT

A long-term state transition is estimated in consideration of variations among individuals. A state transition estimation device acquires a first feature vector of a first individual in a first age range, a second feature vector of a second individual in a second age range, and individual data of the first individual, generates a latent vector representing variation between the first individuals from the individual data, performs mapping to an estimated feature vector in a case where the first individual falls within a second age range by using the latent vector, updates a mapping parameter so that the mapping approaches optimal transport from a probability distribution of the first feature vector to a probability distribution of the second feature vector, and estimates a probability distribution of the estimated feature vector in a case where the individual in the first age range falls within the second age range.

### INCORPORATION BY REFERENCE

This application is based upon and claims the benefit of priority from Japanese patent application No. 2024-194452, filed on Nov. 6, 2024, the disclosure of which is incorporated herein in its entirety by reference.

### TECHNICAL FIELD

The present disclosure relates to a state transition estimation device, a state transition estimation method, and a state transition estimation program.

### BACKGROUND ART

The long-term transition prediction of the disease risk and the transition estimation of the health condition are effective for the future life plan design. By predicting the long-term transition of the health condition and the disease risk from the medical examination result and the daily health activity, the lifetime cost can be predicted, and for example, can be used for asset management support.

In recent years, a large amount of cross-sectional data has been accumulated, and data analysis has become possible in the fields of healthcare and medical care. For example, Patent Literature 1 describes a technique for constructing a health model in which the health condition of a subject changes by age group.

A technique for predicting a long-term transition is also useful in the fields of healthcare and medical care.
Patent Literature 1: Japanese Unexamined Patent Application Publication No. 2016-038626
Non-patent Literature 1: “Neural Optimal Transport” (Korotin, Alexander, Daniil Selikhanovych, and Evgeny Burnaev, The Eleventh International Conference on Learning Representations (Published as a conference paper at ICLR 2023))

### SUMMARY

However, it may be difficult to sufficiently acquire longitudinal data having temporal connection. The present inventor has studied estimating longitudinal data by estimating a transition between distributions based on cross-sectional data collected for each age group and age by using a technique such as optimal transport based on unique knowledge.

As an example of the technology related to the optimal transport, for example, there is a technology in which machine learning and optimal transport described in the Non-patent Literature 1 are combined. According to the technology described in the Non-patent Literature 1, it is possible to give a distribution having a probabilistic spread as a transition destination to data of a certain point of transition source.

As a result, for example, it is possible to stochastically indicate how the test value transitions as the age advances based on the test value of the medical examination of the subject in a certain age group. At first glance, this seems to be able to express a situation in which even if the test value is the same at a certain point in time, the test value does not necessarily transition to the same test value due to variation between individuals. However, in the technique described in the Non-patent Literature 1, since a distribution having a spread is generated by giving noise, it does not reflect actual variation between individuals.

The present disclosure has been made in view of the above problems, and an example object thereof is to provide a technique for estimating a long-term state transition in consideration of variations among individuals.

A state transition estimation device according to an example aspect of the present disclosure includes a data input unit that acquires a first feature vector indicating a state of each of a plurality of first individuals in a first age range, a second feature vector indicating a state of each of a plurality of second individuals in a second age range older than the first age range, and individual data different from the first feature vector of each of the plurality of first individuals, an individual variation generation unit that generates a latent vector representing a variation between the first individuals from the individual data of the first individual, a data mapping unit that performs mapping from the first feature vector of the first individual to an estimated feature vector in a case where the first individual falls within the second age range using the latent vector of the first individual, an update unit that updates a mapping parameter in the data mapping unit based on the first feature vectors and the estimated feature vectors of the plurality of first individuals and the second feature vectors of the plurality of second individuals in such a way that the mapping in the data mapping unit approaches an optimal transport from a probability distribution of the first feature vector to a probability distribution of the second feature vector, and a probability distribution estimation unit that estimates a probability distribution of the estimated feature vector in a case where an individual falls within the second age range from the first feature vector of the individual in the first age range using the data mapping unit in which the mapping parameter is updated by the update unit.

A state transition estimation method according to an example aspect of the present disclosure includes a data input process of acquiring a first feature vector indicating a state of each of a plurality of first individuals in a first age range, a second feature vector indicating a state of each of a plurality of second individuals in a second age range older than the first age range, and individual data different from the first feature vector of each of the plurality of first individuals, an individual variation generation process of generating a latent vector representing a variation between the first individuals from the individual data of the first individual, a data mapping process of performing mapping from the first feature vector of the first individual to an estimated feature vector in a case where the first individual falls within the second age range using the latent vector of the first individual, an update process of updating a mapping parameter applied to the data mapping process based on the first feature vectors and the estimated feature vectors of the plurality of first individuals and the second feature vectors of the plurality of second individuals in such a way that the mapping in the data mapping process approaches an optimal transport from a probability distribution of the first feature vector to a probability distribution of the second feature vector, and a probability distribution estimation process of estimating a probability distribution of the estimated feature vector in a case where an individual falls within the second age range from the first feature vector of the individual in the first age range using the data mapping process in which the mapping parameter is updated by the update process.

A state transition estimation program according to an example aspect of the present disclosure causes a computer to execute a data input process of acquiring a first feature vector indicating a state of each of a plurality of first individuals in a first age range, a second feature vector indicating a state of each of a plurality of second individuals in a second age range older than the first age range, and individual data different from the first feature vector of each of the plurality of first individuals, an individual variation generation process of generating a latent vector representing a variation between the first individuals from the individual data of the first individual, a data mapping process of performing mapping from the first feature vector of the first individual to an estimated feature vector in a case where the first individual falls within the second age range using the latent vector of the first individual, an update process of updating a mapping parameter applied to the data mapping process based on the first feature vectors and the estimated feature vectors of the plurality of first individuals and the second feature vectors of the plurality of second individuals in such a way that the mapping in the data mapping process approaches an optimal transport from a probability distribution of the first feature vector to a probability distribution of the second feature vector, and a probability distribution estimation process of estimating a probability distribution of the estimated feature vector in a case where an individual falls within the second age range from the first feature vector of the individual in the first age range using the data mapping process in which the mapping parameter is updated by the update process.

According to an example aspect of the present disclosure, there is an exemplary effect that a technology for estimating a long-term state transition in consideration of variation between individuals can be provided.

### BRIEF DESCRIPTION OF DRAWINGS

The above and other aspects, features and advantages of the present disclosure will become more apparent from the following description of certain exemplary embodiments when taken in conjunction with the accompanying drawings, in which:

FIG. 1 is a block diagram illustrating a configuration of a state transition estimation device according to the present disclosure;

FIG. 2 is a flowchart illustrating a flow of a state transition estimation method according to the present disclosure;

FIG. 3 is a block diagram illustrating a configuration of a state transition estimation device according to the present disclosure;

FIG. 4 is a diagram illustrating an example of a forecast circle according to the present disclosure;

FIG. 5 is a diagram illustrating an example of a forecast circle according to the present disclosure;

FIG. 6 is a flowchart illustrating a flow of a state transition estimation method according to the present disclosure;

FIG. 7 is a diagram illustrating an example of a forecast circle according to the present disclosure;

FIG. 8 is a diagram illustrating an example of a forecast circle according to the present disclosure;

FIG. 9 is a block diagram illustrating a configuration of a life plan decision making support device according to the present disclosure; and

FIG. 10 is a block diagram illustrating a configuration of a computer that functions as a state transition estimation device according to the present disclosure.

### EXAMPLE EMBODIMENT

Hereinafter, example embodiments will be exemplified. However, the present invention is not limited to exemplary example embodiments described below, and various alterations can be made within the scope described in the claims. For example, example embodiments obtained by appropriately combining technologies (some or all of things or methods) adopted in the following exemplary example embodiments can also be included in the scope of the present invention. Example embodiments obtained by appropriately omitting some of the technologies adopted in the following exemplary example embodiments can also be included in the scope of the present invention. Effects mentioned in the following exemplary example embodiments are examples of effects expected in the exemplary example embodiments, and do not define extension of the present invention. That is, example embodiments that do not achieve the effects mentioned in the exemplary example embodiments to be described below can also be included in the scope of the present invention.

### First Exemplary Example Embodiment

A first exemplary example embodiment, which is an example of an example embodiment, will be described in detail with reference to the drawings. The present exemplary example embodiment is a basic form of each exemplary example embodiment described below. An application range of each technology adopted in the present exemplary example embodiment is not limited to the present exemplary example embodiment. In other words, each technology adopted in the present exemplary example embodiment can also be adopted in another exemplary example embodiment included in the present disclosure within a range in which no particular technical problem occurs. Each technology illustrated in the drawings referred to for describing the present exemplary example embodiment can also be adopted in another exemplary example embodiment included in the present disclosure within a range in which no particular technical problem occurs.

### (Configuration of State Transition Estimation Device)

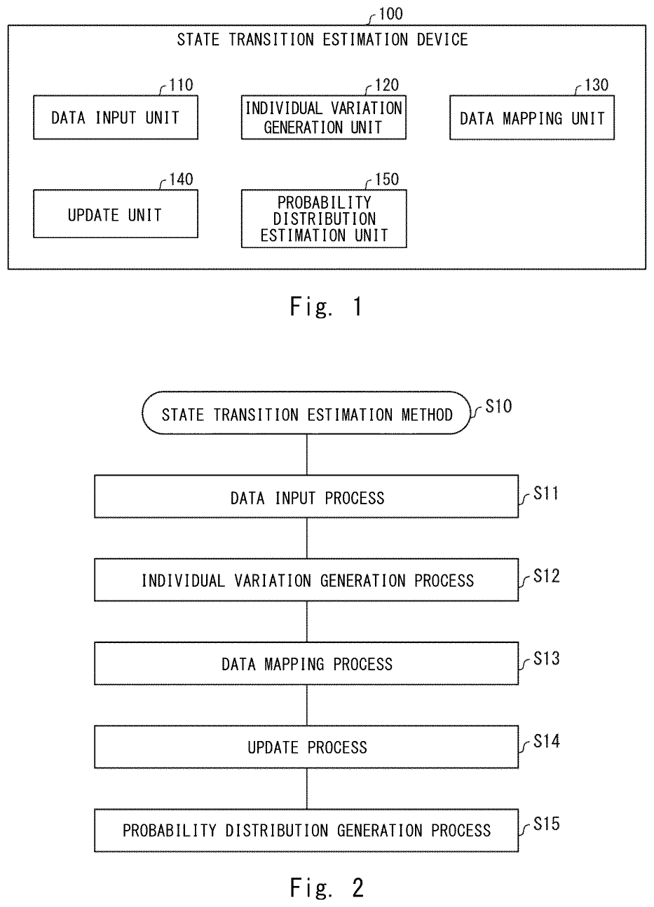

A configuration of a state transition estimation device 100 will be described with reference to FIG. 1. FIG. 1 is a block diagram illustrating the configuration of the state transition estimation device 100. As illustrated in FIG. 1, the state transition estimation device 100 includes a data input unit 110, an individual variation generation unit 120, a data mapping unit 130, an update unit 140, and a probability distribution estimation unit 150.

By receiving an input from the outside or converting an input from the outside, the data input unit 110 acquires a first feature vector indicating a state of each of a plurality of first individuals in a first age range, a second feature vector indicating a state of each of a plurality of second individuals in a second age range older than the first age range, and individual data different from the first feature vector of each of the plurality of first individuals.

The first individual and the second individual may be persons or equipment such as machines. The first individual and the second individual are not necessarily the same, but may be partially the same. The first age range and the second age range are not particularly limited, but may be relevant to, for example, age groups in 10-year increments. As an example, in a case where the first individual and the second individual are persons, the first age range can be 60's, the second age range can be 70's, and the like. In a case where the first individual and the second individual are facilities, the first age range and the second age range may be a range of facility age.

The states indicated by the first feature vector and the second feature vector are not particularly limited, but may be two or more states or three or more states. In a case where the first individual and the second individual are persons, each state may be test value data (HbA1c or the like) such as a medical examination, a risk index value of a disease, life log data (weight, blood pressure, number of steps, and the like), or the like. In a case where the first individual and the second individual are facilities, each state may be test value data such as a periodic test, an output value, power consumption, or the like.

The individual data is data indicating an attribute and a state of each individual different from the state indicated by the first feature vector and the second feature vector, and is not particularly limited. In a case where the first individual is a person, the individual data may include attribute data such as sex and age group, or may include a test value, a disease risk index value, life log data, and the like different from the state indicated by the first feature vector and the second feature vector. In a case where the first individual is a facility, the individual data may include attribute data such as a format and a manufacturer, or may include test value data such as a periodic test, an output value, power consumption, and the like.

The individual variation generation unit 120 generates a latent vector representing a variation among the first individuals from the individual data of the first individuals. The expression of the variation among the first individuals can also be rephrased as indicating the individuality of each of the first individuals, and means that each of the first individuals serves as an index of what kind of feature each of the first individuals has from a viewpoint other than the state indicated by the first feature vector. The method for generating the latent vector is not particularly limited, but any method may be used as long as the individual data is converted into a vector.

The data mapping unit 130 performs mapping from the first feature vector of the first individual to the estimated feature vector in a case where the first individual falls within the second age range by using the latent vector of the first individual. In other words, the data mapping unit 130 performs mapping from the feature space in which the first feature vector is distributed to the feature space in which the second feature vector is distributed.

The update unit 140 updates the mapping parameter in the data mapping unit 130 based on the first feature vectors and the estimated feature vectors of the plurality of first individuals and the second feature vectors of the plurality of second individuals so that the mapping in the data mapping unit 130 approaches the optimal transport from the probability distribution of the first feature vector to the probability distribution of the second feature vector. For example, the update unit 140 calculates the transport cost from the first feature vector to the estimated feature vector, and repeats updating the mapping parameter so as to reduce the transport cost, thereby approaching the optimal transport.

The probability distribution estimation unit 150 estimates the probability distribution of the estimated feature vector in a case where the individual falls within the second age range from the first feature vector of the individual in the first age range using the data mapping unit 130 in which the mapping parameter is updated by the update unit 140. The individual may be an individual different from the first individual or may be the first individual.

### (Effect of State Transition Estimation Device)

As described above, the state transition estimation device 100 employs a configuration in which the latent vector generated from the individual data of the first individual is used to perform mapping from the first feature vector of the first individual to the estimated feature vector in a case where the first individual falls within the second age range. Therefore, according to the state transition estimation device 100, it is possible to obtain an effect of being able to estimate a long-term state transition in consideration of variations among individuals.

### (Flow of State Transition Estimation Method)

A flow of a state transition estimation method S10 will be described with reference to FIG. 2. FIG. 2 is a flowchart illustrating the flow of the state transition estimation method S10. As illustrated in FIG. 2, the state transition estimation method S10 includes a data input process S11, an individual variation generation process S12, a data mapping process S13, an update process S14, and a probability distribution estimation process S15.

The data input process S11 acquires a first feature vector indicating a state of each of a plurality of first individuals in a first age range, a second feature vector indicating a state of each of a plurality of second individuals in a second age range older than the first age range, and individual data different from the first feature vector of each of the plurality of first individuals.

The individual variation generation process S12 generates a latent vector representing a variation between the first individuals from the individual data of the first individuals.

The data mapping process S13 performs mapping from the first feature vector of the first individual to the estimated feature vector in a case where the first individual falls within the second age range using the latent vector of the first individual.

The update process S14 updates the mapping parameter applied to the data mapping process based on the first feature vectors and the estimated feature vectors of the plurality of first individuals and the second feature vectors of the plurality of second individuals such that the mapping in the data mapping process S13 approaches the optimal transport from the probability distribution of the first feature vector to the probability distribution of the second feature vector.

The probability distribution estimation process S15 estimates the probability distribution of the estimated feature vector in a case where the individual falls within the second age range from the first feature vector of the individual in the first age range using the data mapping process S13 in which the mapping parameter is updated by the update process S14.

### (Effect of State Transition Estimation Method)

As described above, the state transition estimation method S10 employs a configuration in which the latent vector generated from the individual data of the first individual is used to perform mapping from the first feature vector of the first individual to the estimated feature vector in a case where the first individual falls within the second age range. Therefore, according to the state transition estimation method S10, it is possible to obtain an effect of being able to estimate a long-term state transition in consideration of variations among individuals.

### Second Exemplary Example Embodiment

A second exemplary example embodiment, which is an example of an example embodiment, will be described in detail with reference to the drawings. Constituents having the same functions as the constituents described in the above-described exemplary example embodiment are denoted by the same reference sign, and the description thereof will be omitted as appropriate. An application range of each technology adopted in the present exemplary example embodiment is not limited to the present exemplary example embodiment. In other words, each technology adopted in the present exemplary example embodiment can also be adopted in another exemplary example embodiment included in the present disclosure within a range in which no particular technical problem occurs. Each technique illustrated in each of the drawings referred to for describing the present exemplary example embodiment can be employed in the other exemplary example embodiments included in the present disclosure within the scope in which no particular technical problem occurs.

Hereinafter, an example in which the first individual and the second individual are persons and the first feature vector and the second feature vector indicate a state related to health or disease risk will be assumed, but the present example embodiment is not limited thereto. Examples of the state related to health or disease risk include test value data (such as HbA1c) such as a medical examination, a risk index value of a disease, life log data (weight, blood pressure, number of steps, etc.), and the like.

### (Configuration of State Transition Estimation Device)

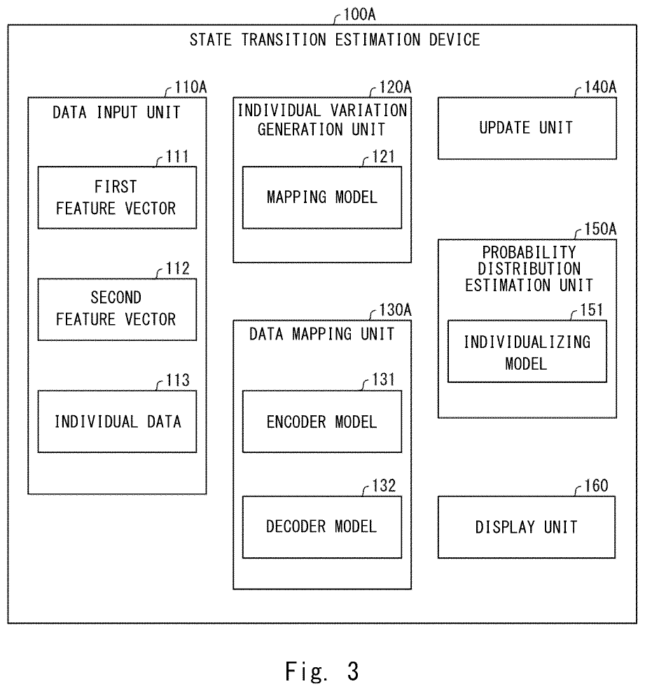

A configuration of a state transition estimation device 100A will be described with reference to FIG. 3. FIG. 3 is a block diagram illustrating the configuration of the state transition estimation device 100A. The state transition estimation device 100A includes a data input unit 110A, an individual variation generation unit 120A, a data mapping unit 130A, an update unit 140A, a probability distribution estimation unit 150A, and a display unit 160.

The data input unit 110A acquires and stores a first feature vector 111, a second feature vector 112, and individual data 113. The first feature vector 111 is a vector, for each first individual, having components relevant to a plurality of states of the first individual, and is described as x₁, . . . , and x_{N} (N is the number of first individuals) in the following description. The second feature vector 112 is a vector having components relevant to a plurality of states of the second individual for each second individual, and is described as y₁, . . . , and y_{N} (N is the number of second individuals) in the following description. In the following description, a case where both the number of first individuals and the number of second individuals are N will be described, but the present example embodiment is not limited thereto. The individual data 113 is data indicating a state and an attribute of the first individual different from those of the first feature vector 111, and is described as z₁, . . . , and z_{N} (N is the number of first individuals) in the following description.

The individual variation generation unit 120A generates a latent vector w from the individual data 113 for each first individual using a mapping model (first machine learning model) 121 that is a machine learning model having the individual data 113 as an input and the latent vector as an output. The number of components of the latent vector w is, for example, the same as the number of components of the first feature vector 111. However, the number of components of the latent vector w may be different from the number of components of the first feature vector 111 as long as calculation in the data mapping unit 130A to be described later is possible. The mapping model 121 may include, for example, a neural network (NN) such as a fully-connected multilayer (FC).

The data mapping unit 130A performs mapping using an encoder model 131 and a decoder model 132 constituting a machine learning model of a variational auto encoder (VAE) type. The data mapping unit 130A inputs the first feature vector x of the first individual to the encoder model 131, and generates an average μ and a variance σ² of the estimated feature vector y\hat{ } obtained by the mapping. Then, the data mapping unit 130A multiplies the variance σ² by the latent vector w (calculates a Hadamard product), inputs the average μ and the variance σ²·latent vector w to the decoder model 132, and outputs the mapped estimated feature vector y\hat{ }.

As described above, in the present example embodiment, the average μ and the variance σ² are generated from the first feature vector x using the machine learning model. On the other hand, in the technique described in the Non-patent Literature 1, the variance σ² is calculated by adding noise sampled from the normal distribution to the first feature vector x to generate a plurality of y\hat{ }. However, in the present example embodiment, since the latent vector w is calculated from the individual data z, only one latent vector w is generated for one first feature vector x. Therefore, since the variance σ² cannot be calculated by generating a plurality of y\hat{ } as in the Non-patent Literature 1, in the present example embodiment, the average μ and the variance σ² are directly generated from the first feature vector x using the machine learning model.

The encoder model 131 and the decoder model 132 may be constituted by, for example, a neural network (NN) such as a fully-connected multilayer (FC).

The update unit 140A calculates the transport cost from the first feature vector x, the second feature vector y, and the estimated feature vector y\hat{ }, and performs update to reduce the transport cost. As the transport cost, the L2 distance may be used similarly to general optimal transport, or other distances may be used. In order to obtain a broad distribution, the restriction of the optimal transport may be relaxed by adding a regularization term. As the regularization term, an entropy term may be added as a general method, or a variation term may be added. Hereinafter, a case using the variation term will be described. As the variance, the variance σ² generated in the data mapping unit 130A is used.

Then, in one example, the parameters (mapping parameters) of the mapping model 121, the encoder model 131, and the decoder model 132 are updated so as to reduce the transport loss which is a loss function defined for minimizing the transport cost. As a result, the parameters (mapping parameters) of the mapping model 121, the encoder model 131, and the decoder model 132 can be updated such that the mapping in the data mapping unit 130A approaches the optimal transport from the probability distribution of the first feature vector x to the probability distribution of the second feature vector y.

Furthermore, in one example, the update unit 140A may further calculate one or more loss functions such as VAE loss, variance loss, and average loss, and update the parameters (mapping parameters) of the mapping model 121, the encoder model 131, and the decoder model 132 so as to minimize the loss function.

The VAE loss is used in a general VAE, and is a loss function for minimizing a square error between an input and an output of the VAE. The variance loss is a loss function for minimizing a square error between the average of the variance σ² output from the encoder model 131 and the variance over the entire data of the estimated feature vector y\hat{ } output from the decoder model 132. The average loss is a loss function for minimizing a square error between the average μ output from the encoder model 131 and the estimated feature vector y\hat{ } output from the decoder model 132. If the estimated feature vector y\hat{ } output by the decoder model 132 is within 1σ from the average μ output by the encoder model 131, the condition may be relaxed such that there is no penalty.

The update unit 140A may further calculate the individual loss and update the parameter of an individualizing model 151 so as to minimize the loss function. Details will be described later.

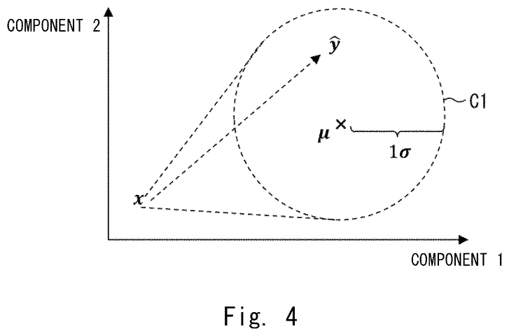

Next, the probability distribution estimation unit 150A will be described. As illustrated in FIG. 4, the probability distribution estimation unit 150A estimates a Gaussian distribution type first probability distribution P(y|x) from the average μ and the variance σ² generated by the data mapping unit 130A. The first probability distribution indicates a range of the estimated feature vector y\hat{ } having a variation of 1σ in a case where the individuality of the first individual is not considered. In the present specification, the range is also referred to as a forecast circle C1.

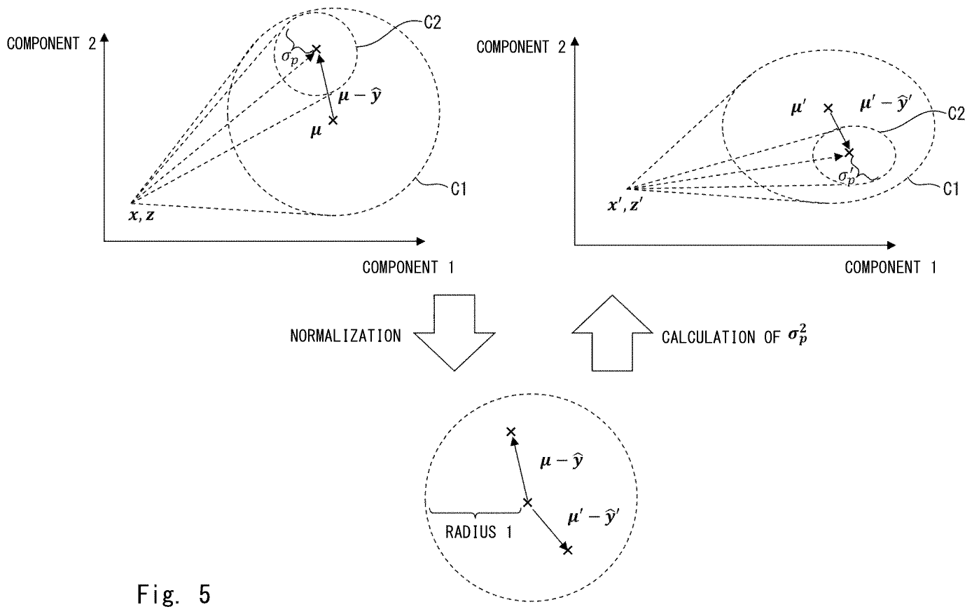

As illustrated in the upper part of FIG. 5, the probability distribution estimation unit 150A may estimate a second probability distribution P(y|z, z) having a variance σₚ² reflecting the individuality of the first individual based on individual data z. The second probability distribution indicates a range (forecast circle C2) more limited than the first probability distribution.

First, the probability distribution estimation unit 150A generates a difference vector μ−y\hat{ } from the individual data z by using the individualizing model 151 that is a machine learning model having the individual data z as an input and the difference vector μ−y\hat{ } as an output. The difference vector μ−y\hat{ } is a difference vector between the estimated feature vector y\hat{ } obtained by the data mapping unit 130A and the average μ of the estimated feature vector y\hat{ }.

The individualizing model 151 may be composed of, for example, a neural network (NN) such as a fully-connected multilayer (FC). The update unit 140A can perform machine learning on the individualizing model 151 by updating the parameter of the individualizing model 151 so as to minimize the square error between the output of the individualizing model 151 and μ−−y\hat{ }.

As illustrated in the upper part of FIG. 5, if the first feature vector x is different (in the upper part of FIG. 5, x and x′ indicate first feature vectors different from each other), the size of the forecast circle C1 of 1σ changes. Therefore, as illustrated in the lower part of FIG. 5, the update unit 140A can absorb the difference and perform machine learning by performing normalization such that the radius is 1.

Then, the probability distribution estimation unit 150A calculates the variance σₚ² according to the ratio of the radius σ of the forecast circle based on the square error of the difference vector μ−y\hat{ } generated from the individual data z using the machine-learned individualizing model 151.

Here, the variance σₚ² is obtained by multiplying the prediction variance, which is the average of the square errors obtained by the learning after normalization, by the radius σ of the forecast circle to return the normalization.

As a result, the probability distribution estimation unit 150A can estimate the Gaussian distribution type second probability distribution P(y|z, z) with the mapped estimated feature vector y\hat{ } as an average and σₚ² as a variance. The second probability distribution indicates a range of the estimated feature vector y\hat{ } reflecting the individuality of the first individual. In the present specification, the range is also referred to as a forecast circle C2.

The display unit 160 includes a display device or the like. The display unit 160 displays the forecast circle C1 indicating the first probability distribution estimated by the probability distribution estimation unit 150A and the forecast circle C2 indicating the second probability distribution.

### (Flow of State Transition Estimation Method)

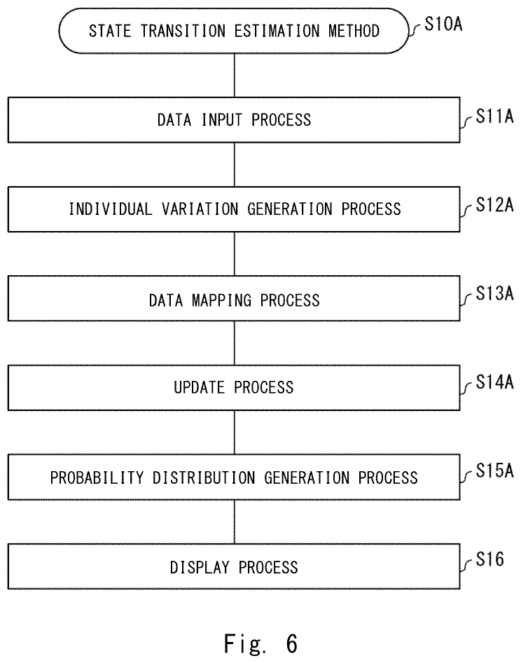

A flow of a state transition estimation method S10A will be described with reference to FIG. 6. FIG. 6 is a flowchart illustrating a flow of the state transition estimation method S10A. As illustrated in FIG. 6, the state transition estimation method S10A includes a data input process S11A, an individual variation generation process S12A, a data mapping process S13A, an update process S14A, a probability distribution estimation process S15A, and a display process S16.

In step S11A, the data input unit 110A acquires the first feature vectors x₁, . . . , and x_{N}, and sequentially provides the first feature vectors xₖ (1≤k≤N) to the data mapping unit 130A and the update unit 140A. The data input unit 110A acquires the second feature vectors y₁, . . . , and y_{N}, and provides each of the second feature vectors yₖ (1≤k≤N) to the update unit 140A. The data input unit 110A acquires the individual data z₁, . . . , and z_{N}, and sequentially provides each individual data zₖ (1≤k≤N) to the individual variation generation unit 120A.

In step S12A, the individual variation generation unit 120A generates the latent vector w from the individual data zₖ provided from the data input unit 110A using the mapping model 121, and provides the latent vector w to the data mapping unit 130A.

In step S13A, the data mapping unit 130A inputs the first feature vector xₖ provided from the data input unit 110A to the encoder model 131, and generates the average μ_{φ} and the variance σ_{φ}². The data mapping unit 130A obtains the following value (1) by multiplying the variance σ_{φ}² by w provided from the individual variation generation unit 120A (calculating the Hadamard product) and adding the average μ_{φ}.

$$\begin{matrix}[ Math . 1 ] & \\ {μ}_{ϕ} + {σ}_{ϕ}^{2} ⊙ z & ( 1 )\end{matrix}$$

The data mapping unit 130A inputs the obtained value to the decoder model 132 and calculates the estimated feature vector y\hat{ }ₖ. Then, the data mapping unit 130A provides the average μ_{φ}, the variance σ_{φ}², and the estimated feature vector y\hat{ }ₖ to the update unit 140A.

In step S14A, the update unit 140A calculates a transport loss, a VAE loss, a variance loss, an average loss, and an individual loss, and updates parameters of each model.

First, the transport loss will be described. The update unit 140A calculates the following value (2) from the first feature vector xₖ provided from the data input unit 110A and the estimated feature vector y\hat{ }ₖ and the variance σ_{φ}² provided from the data mapping unit 130A.

$$\begin{matrix}[ Math . 2 ] & \\ \vert ( {x}_{k} - {\hat{y}}_{k} ) {\vert}^{2} - γ & ( 2 )\end{matrix}$$

The update unit 140A calculates k=1 to N and averages the result to obtain the following value (3).

$$\begin{matrix}[ Math . 3 ] & \\ \frac{1}{N} {\sum }_{k}^{N} \vert ( {x}_{k} - {\hat{y}}_{k} ) {\vert}^{2} - γ & ( 3 )\end{matrix}$$

The update unit 140A inputs the estimated feature vector y\hat{ }ₖ provided from the data mapping unit 130A to the function f, calculates the obtained f(y\hat{ }ₖ) for k=1 to N, and averages the f to obtain the following value (4).

$$\begin{matrix}[ Math . 4 ] & \\ \frac{1}{N} {\sum }_{k}^{N} f ( {\hat{y}}_{k} ) & ( 4 )\end{matrix}$$

Then, the update unit 140A calculates the following transport loss L₁ from the value (3) and the value (4).

$$\begin{matrix}[ Math . 5 ] & \\ {ℒ}_{2} = \frac{1}{N} {\sum }_{k}^{N} \vert ( {x}_{k} - {\hat{y}}_{k} ) {\vert}^{2} - γ {σ}_{ϕ}^{2} -- \frac{1}{N} {\sum }_{k}^{N} f ( {\hat{y}}_{k} ) & \end{matrix}$$

The update unit 140A inputs the second feature vector yₖ provided from the data input unit 110A to the function f, calculates the obtained f(yₖ) for k=1 to N, and averages the f(yₖ) to obtain the following value (5).

$$\begin{matrix}[ Math . 6 ] & \\ \frac{1}{N} {\sum }_{k}^{N} f ( {y}_{k} ) & ( 5 )\end{matrix}$$

Then, the update unit 140A calculates the following transport loss L₂ from the value (4) and the value (5).

$$\begin{matrix}[ Math . 7 ] & \\ {ℒ}_{2} = \frac{1}{N} {Σ}_{k}^{N} f ( {\hat{y}}_{k} ) - \frac{1}{N} {Σ}_{k}^{N} f ( {y}_{k} ) & \end{matrix}$$

The update unit 140A updates the parameters (mapping parameters) of the mapping model, the encoder model, and the decoder model so that the transport loss L₁ becomes small and the transport loss L₂ becomes large.

Next, the VAE loss will be described. The update unit 140A calculates a square error between the second feature vector yₖ provided from the data input unit 110A and the estimated feature vector y\hat{ }ₖ provided from the data mapping unit 130A as the VAE loss, and updates the parameters (mapping parameters) of the mapping model, the encoder model, and the decoder model so as to reduce the VAE loss.

Next, the variance loss will be described. The update unit 140A calculates, as the variance loss, a square error between the average of the variance σ² provided from the data mapping unit 130A and the variance of the estimated feature vector y\hat{ } provided from the data mapping unit 130A over k=1 to N, and updates the parameters (mapping parameters) of the mapping model, the encoder model, and the decoder model so as to reduce the variance loss.

Subsequently, the average loss will be described. The update unit 140A calculates, as the average loss, a square error between the average of the average μ_{φ} provided from the data mapping unit 130A and the average of the estimated feature vector y\hat{ }ₖ provided from the data mapping unit 130A over k=1 to N. If the estimated feature vector y\hat{ }ₖ is within 1σ_{φ} from the average μ_{φ}, the condition may be loosened so that there is no penalty.

Subsequently, the individual loss will be described. The update unit 140A calculates, as the individual loss, a square error between the estimated value of the difference vector μ−y\hat{ } generated by the probability distribution estimation unit 150A using the individualizing model 151 and the difference vector between the actual average po and the estimated feature vector y\hat{ }ₖ, and updates the parameter of the individualizing model 151 so as to minimize the individual loss.

As described above, the update unit 140A updates the parameters of each model (learns each model). In one example, each model may be optimized by repeating steps S12A to S14A.

In step S15A, the probability distribution estimation unit 150A estimates a Gaussian distribution type first probability distribution P(y|x) from the average μ_{φ} and the variance σ_{φ}² provided from the data mapping unit 130A. In step S16, the display unit 160 illustrates the forecast circle C1 in the range 1σ_{φ} relevant to the first probability distribution.

In step S15A, the probability distribution estimation unit 150A calculates a variance σₚ² reflecting the individuality from the individual data zₖ and the first feature vector xₖ using the individualizing model 151, and estimates a Gaussian distribution type second probability distribution P(y|x, z) in which the estimated feature vector y\hat{ }ₖ is an average and σₚ² is a variance. In step S16, the display unit 160 illustrates a more limited forecast circle C2 in the range 11σₚ relevant to the second probability distribution.

### Third Exemplary Example Embodiment

A third exemplary example embodiment, which is an example of an example embodiment, will be described in detail with reference to the drawings. Constituents having the same functions as the constituents described in the above-described exemplary example embodiment are denoted by the same reference sign, and the description thereof will be omitted as appropriate. An application range of each technology adopted in the present exemplary example embodiment is not limited to the present exemplary example embodiment. In other words, each technology adopted in the present exemplary example embodiment can also be adopted in another exemplary example embodiment included in the present disclosure within a range in which no particular technical problem occurs. Each technique illustrated in each of the drawings referred to for describing the present exemplary example embodiment can be employed in the other exemplary example embodiments included in the present disclosure within the scope in which no particular technical problem occurs.

In the present example embodiment, at least some of the first individuals are relevant to at least some of the second individuals. The correspondence between the first individual and the second individual means a relationship in which the aged first individual is the second individual, and in a case where the first individual and the second individual are persons, means a relationship in which the first individual and the second individual are the same person.

At this time, in the calculation of the transport loss, the VAE loss, the variance loss, the average loss, and the individual loss described above, the update unit 140A can further improve the accuracy by calculating an additional loss function using y˜ that is the second feature vector y of the second individual relevant to the first individual instead of the estimated feature vector y\hat{ } in addition to calculating various losses using the estimated feature vector y\hat{ }.

Furthermore, the update unit 140A may update the parameters (mapping parameters) of the mapping model 121, the encoder model 131, and the decoder model 132 such that the estimated feature vector y\hat{ } mapped from the first feature vector x of the first individual having the relevant second individual and y˜ that is the second feature vector y of the second individual relevant to the first individual approach each other.

For example, the update unit 140A may calculate, as the mapping loss, a difference (for example, a square error) between the estimated feature vector y\hat{ } mapped from the first feature vector x of the first individual in which the relevant second individual exists by the data mapping unit 130A and y˜, which is the second feature vector y of the second individual relevant to the first individual, and update the parameters (mapping parameters) of the mapping model 121, the encoder model 131, and the decoder model 132 so as to reduce the mapping loss.

For example, the update unit 140A may have a machine learning model in which the data mapping unit 130A learns the relationship between the estimated feature vector y\hat{ } mapped from the first feature vector x of the first individual in which the relevant second individual exists and y˜ that is the second feature vector y of the second individual relevant to the first individual. Then, the update unit 140A may calculate an R2 loss, which is a loss function for reducing the R2 score of the machine learning model, and update the parameters (mapping parameters) of the mapping model 121, the encoder model 131, and the decoder model 132 so as to reduce the R2 loss.

### Fourth Exemplary Example Embodiment

A fourth exemplary example embodiment, which is an example of an example embodiment, will be described in detail with reference to the drawings. Constituents having the same functions as the constituents described in the above-described exemplary example embodiment are denoted by the same reference sign, and the description thereof will be omitted as appropriate. An application range of each technology adopted in the present exemplary example embodiment is not limited to the present exemplary example embodiment. In other words, each technology adopted in the present exemplary example embodiment can also be adopted in another exemplary example embodiment included in the present disclosure within a range in which no particular technical problem occurs. Each technique illustrated in each of the drawings referred to for describing the present exemplary example embodiment can be employed in the other exemplary example embodiments included in the present disclosure within the scope in which no particular technical problem occurs.

The state transition predicted by the state transition estimation device 100A is not limited to one stage, and two or more stages may be predicted. For example, in a case where the first individual is an individual in his/her 60's, three or more feature vectors such as 70's and 80's may be estimated. The unit is not limited to 10 years. The first feature vector of the first individual is denoted by x, the second feature vector of the second individual is denoted by y₁, and the third feature vector of the third individual is denoted by y₂.

The probability distribution estimation unit 150A first estimates the average μ₁, the variance σ₁², and the probability distribution P(y₁|x) of the estimated feature vectors in the age range relevant to the second individual from the first feature vector x of the first individual. Further, the probability distribution estimation unit 150A estimates an average μ₂, a variance σ₂², and a probability distribution P(y₂|y₁) of the estimated feature vectors in the age range relevant to the third individual from the second feature vector y₁ of the second individual.

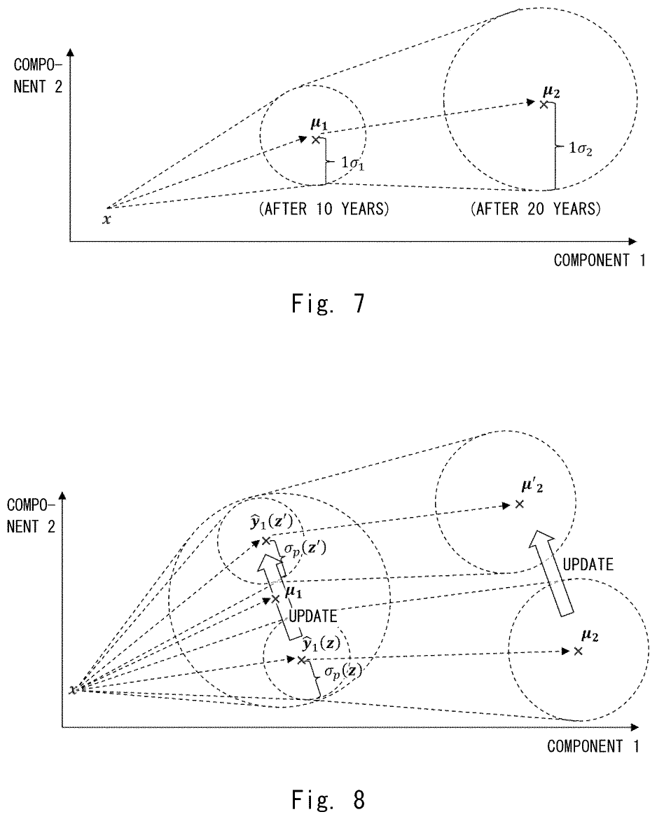

As illustrated in FIG. 7, the probability distribution estimation unit 150A may obtain an average μ₁ and a variance σ₁² in a case where x is given to the probability distribution P(y₁|x), and the display unit 160 may display the relevant forecast circle. Further, the probability distribution estimation unit 150A may obtain an average μ₂ and a variance σ₂² in a case where μ₁ is given to the probability distribution P(y₂|y₁), and the display unit 160 may display the relevant forecast circle. As in the example described above, in a case where x is a value at the age of 60, μ₁ can be interpreted as a transition prediction after 10 years, and μ₂ can be interpreted as a transition prediction after 20 years.

In a case where the individual data z further includes daily changing life log data such as the average number of steps and the average weight in units of weeks, the probability distribution estimation unit 150A first estimates a probability distribution P(y₁|x, z) in which the average is y\hat{ }₁ and the variance is σₚ² using the individual data z collected in the same period as the first feature vector x. The display unit 160 displays a forecast circle using y\hat{ }₁ and σₚ². Further, the probability distribution estimation unit 150A estimates P(y₂|y₁), obtains an average μ₂ and a variance σ₂² in a case where y\hat{ }₁ is given, and the display unit 160 displays the relevant forecast circle.

Then, the probability distribution estimation unit 150A further obtains an average μ′₂ and a variance σ_{2′}², for example, in a case where the average y\hat{ }₁(z′), the variance σₚ²(z′), and y\hat{ }₁(z′) are given in response to the change in the individual data z, and the display unit 160 displays the relevant forecast circle.

At this time, in order to recognize the change from the previous state, the display unit 160 displays the change in the forecast circle, such as animation display of a new forecast circle. As described above, in a case where the individual data includes data that can change on the time axis, the probability distribution estimation unit 150A may estimate the probability distribution of the estimated feature vector again in response to the change in the individual data, and the display unit 160 may display the change in the probability distribution of the estimated feature vector estimated by the probability distribution estimation unit 150A. By automatically and in real time performing the timing detection and the update, it is possible to expect an effect of encouraging healthy behavior related to the life log.

### Fifth Exemplary Example Embodiment

A fifth exemplary example embodiment, which is an example of an example embodiment, will be described in detail with reference to the drawings. Constituents having the same functions as the constituents described in the above-described exemplary example embodiment are denoted by the same reference sign, and the description thereof will be omitted as appropriate. An application range of each technology adopted in the present exemplary example embodiment is not limited to the present exemplary example embodiment. In other words, each technology adopted in the present exemplary example embodiment can also be adopted in another exemplary example embodiment included in the present disclosure within a range in which no particular technical problem occurs. Each technique illustrated in each of the drawings referred to for describing the present exemplary example embodiment can be employed in the other exemplary example embodiments included in the present disclosure within the scope in which no particular technical problem occurs.

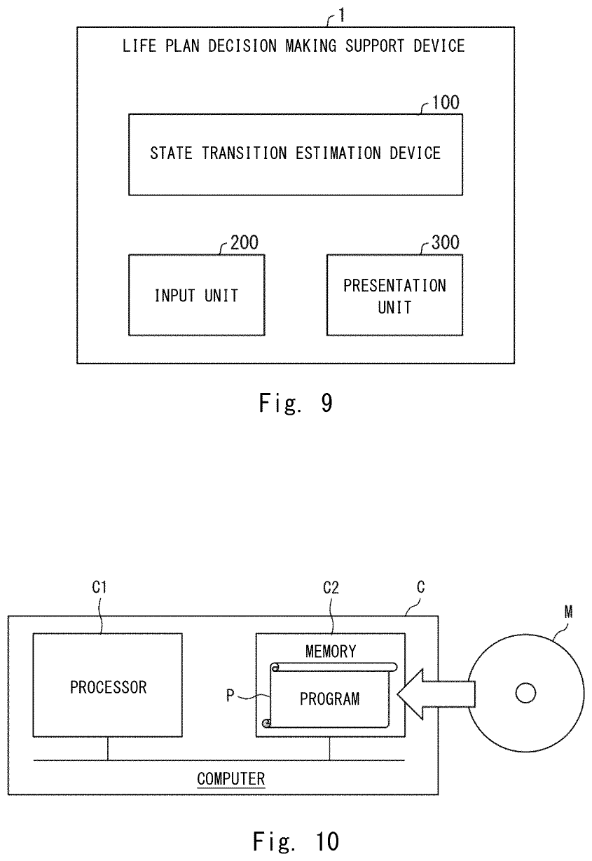

As illustrated in FIG. 9, a life plan decision making support device 1 includes a state transition estimation device 100, an input unit 200, and a presentation unit 300. In a case where the user inputs the state related to the health or disease risk of the user and other individual data via the input unit 200, the state transition estimation device 100 may predict the state related to the health or disease risk of the user in the future, and the presentation unit 300 may present an asset management proposal or the like based on the prediction of the lifetime cost to the user. As a result, it is possible to support the decision making on the life plan of the user.

### [Achievement Example by Software]

Some or all of the functions of the state transition estimation devices 100 and 100A (hereinafter, also referred to as “each of the above-described devices”) may be implemented by hardware such as an integrated circuit (IC chip) or may be implemented by software.

In the latter case, each of the above devices is achieved by, for example, a computer that executes instructions of a program as software for achieving each function. An example of such a computer (hereinafter, referred to as a computer C) is illustrated in FIG. 10. FIG. 10 is a block diagram illustrating a hardware configuration of a computer C functioning as each of the above devices.

The computer C includes at least one processor C1 and at least one memory C2. A program P causing the computer C to operate as each of the above devices is recorded in the memory C2. In the computer C, by the processor C1 reading the program P from the memory C2 and executing the program P, each function of each of the above devices is achieved.

As the processor C1, for example, a Central Processing Unit (CPU), a Graphic Processing Unit (GPU), a Digital Signal Processor (DSP), a Micro Processing Unit (MPU), a Floating point number Processing Unit (FPU), a Physics Processing Unit (PPU), a Tensor Processing Unit (TPU), a quantum processor, a microcontroller, or a combination of these can be used. As the memory C2, for example, a flash memory, a Hard Disk Drive (HDD), a Solid State Drive (SSD), or a combination of these can be used.

The computer C may further include a Random Access Memory (RAM) for loading the program P at the time of execution and temporarily storing various types of data. The computer C may further include a communication interface for transmitting and receiving data to and from another device. The computer C may further include an input/output interface for connecting input/output devices such as a keyboard, a mouse, a display, and a printer.

The program P can be recorded in a non-transitory tangible recording medium M readable by the computer C. As such a recording medium M, for example, a tape, a disk, a card, a semiconductor memory, or a programmable logic circuit can be used. The computer C can acquire the program P via such a recording medium M. The program P can be transmitted via a transmission medium. As such a transmission medium, for example, a communication network or a broadcast wave can be used. The computer C can also acquire the program P via such a transmission medium.

Each of the above functions of each of the above devices may be achieved by a single processor provided in a single computer, may be achieved in cooperation with a plurality of processors provided in a single computer, or may be achieved in cooperation with a plurality of processors provided in a plurality of computers. The program for causing each of the above devices to achieve each of the above functions may be stored in a single memory provided in a single computer, may be stored in a distributed manner in a plurality of memories provided in a single computer, or may be stored in a distributed manner in a plurality of memories provided in a plurality of computers.

### [Supplementary Information 1]

The whole or part of the example embodiments disclosed above can be described as, but not limited to, the following supplementary notes.

### (Supplementary Note 1)

A state transition estimation device including:
a data input unit that acquires a first feature vector indicating a state of each of a plurality of first individuals in a first age range, a second feature vector indicating a state of each of a plurality of second individuals in a second age range older than the first age range, and individual data different from the first feature vector of each of the plurality of first individuals;
an individual variation generation unit that generates a latent vector representing a variation between the first individuals from the individual data of the first individual;
a data mapping unit that performs mapping from the first feature vector of the first individual to an estimated feature vector in a case where the first individual falls within the second age range using the latent vector of the first individual;
an update unit that updates a mapping parameter in the data mapping unit based on the first feature vectors and the estimated feature vectors of the plurality of first individuals and the second feature vectors of the plurality of second individuals in such a way that the mapping in the data mapping unit approaches an optimal transport from a probability distribution of the first feature vector to a probability distribution of the second feature vector; and
a probability distribution estimation unit that estimates a probability distribution of the estimated feature vector in a case where an individual falls within the second age range from the first feature vector of the individual in the first age range using the data mapping unit in which the mapping parameter is updated by the update unit.

### (Supplementary Note 2)

The state transition estimation device according to Supplementary Note 1, in which the data mapping unit outputs an average and a variance of the estimated feature vector obtained by the mapping using a machine learning model of a variational auto encoder (VAE) type.

### (Supplementary Note 3)

The state transition estimation device according to Supplementary Note 2, in which the probability distribution estimation unit estimates a probability distribution of the estimated feature vector in a case where an individual falls within the second age range from the individual data of the individual in the first age range and the first feature vector using a machine learning model having the individual data as an input and a difference vector between the estimated feature vector obtained by the data mapping unit and an average of the estimated feature vector as an output.

### (Supplementary Note 4)

The state transition estimation device according to Supplementary Note 3, in which
the probability distribution estimation unit estimates a first probability distribution of the estimated feature vector from the first feature vector of an individual in the first age range and estimates a second probability distribution of the estimated feature vector from the individual data of an individual in the first age range and the first feature vector, and
the state transition estimation device further includes a display unit that displays the first probability distribution and the second probability distribution.

### (Supplementary Note 5)

The state transition estimation device according to Supplementary Note 1, in which
at least some of the first individuals and at least some of the second individuals are relevant to each other, and
the update unit updates the mapping parameter in the data mapping unit in such a way that the estimated feature vector mapped from the first feature vectors of the at least some of the first individuals and the second feature vector of the at least some of the second individuals approach each other in the data mapping unit.

### (Supplementary Note 6)

The state transition estimation device according to Supplementary Note 1, in which
the individual data includes data that can change on a time axis,
the probability distribution estimation unit estimates a probability distribution of the estimated feature vector again in response to a change in the individual data, and
the state transition estimation device further includes a display unit that displays a change in the probability distribution of the estimated feature vector estimated by the probability distribution estimation unit.

### (Supplementary Note 7)

The state transition estimation device according to any one of Supplementary Notes 1 to 7, in which
the first individual, the second individual, and the individual are persons, and
the first feature vector, the second feature vector, and the estimated feature vector indicate a state related to health or a disease risk.

### (Supplementary Note 8)

A life plan decision making support device including the state transition estimation device according to Supplementary Note 7.

### (Supplementary Note 9)

A state transition estimation method including:
a data input process of acquiring a first feature vector indicating a state of each of a plurality of first individuals in a first age range, a second feature vector indicating a state of each of a plurality of second individuals in a second age range older than the first age range, and individual data different from the first feature vector of each of the plurality of first individuals;
an individual variation generation process of generating a latent vector representing a variation between the first individuals from the individual data of the first individual;
a data mapping process of performing mapping from the first feature vector of the first individual to an estimated feature vector in a case where the first individual falls within the second age range using the latent vector of the first individual;
an update process of updating a mapping parameter applied to the data mapping process based on the first feature vectors and the estimated feature vectors of the plurality of first individuals and the second feature vectors of the plurality of second individuals in such a way that the mapping in the data mapping process approaches an optimal transport from a probability distribution of the first feature vector to a probability distribution of the second feature vector; and
a probability distribution estimation process of estimating a probability distribution of the estimated feature vector in a case where an individual falls within the second age range from the first feature vector of the individual in the first age range using the data mapping process in which the mapping parameter is updated by the update process.

### (Supplementary Note 10)

A non-transitory computer readable medium having stored therein a state transition estimation program causing a computer to execute:
a data input process of acquiring a first feature vector indicating a state of each of a plurality of first individuals in a first age range, a second feature vector indicating a state of each of a plurality of second individuals in a second age range older than the first age range, and individual data different from the first feature vector of each of the plurality of first individuals;
an individual variation generation process of generating a latent vector representing a variation between the first individuals from the individual data of the first individual;
a data mapping process of performing mapping from the first feature vector of the first individual to an estimated feature vector in a case where the first individual falls within the second age range using the latent vector of the first individual;
an update process of updating a mapping parameter applied to the data mapping process based on the first feature vectors and the estimated feature vectors of the plurality of first individuals and the second feature vectors of the plurality of second individuals in such a way that the mapping in the data mapping process approaches an optimal transport from a probability distribution of the first feature vector to a probability distribution of the second feature vector; and
a probability distribution estimation process of estimating a probability distribution of the estimated feature vector in a case where an individual falls within the second age range from the first feature vector of the individual in the first age range using the data mapping process in which the mapping parameter is updated by the update process.

### [Supplementary Information 2]

The whole or part of the example embodiments disclosed above can be described as, but not limited to, the following supplementary notes.

### (Supplementary Note 1)

A state transition estimation device including at least one processor,
wherein the at least one processor executes:
a data input process of acquiring a first feature vector indicating a state of each of a plurality of first individuals in a first age range, a second feature vector indicating a state of each of a plurality of second individuals in a second age range older than the first age range, and individual data different from the first feature vector of each of the plurality of first individuals;
an individual variation generation process of generating a latent vector representing a variation between the first individuals from the individual data of the first individual;
a data mapping process of performing mapping from the first feature vector of the first individual to an estimated feature vector in a case where the first individual falls within the second age range using the latent vector of the first individual;
an update process of updating a mapping parameter applied to the data mapping process based on the first feature vectors and the estimated feature vectors of the plurality of first individuals and the second feature vectors of the plurality of second individuals in such a way that the mapping in the data mapping process approaches an optimal transport from a probability distribution of the first feature vector to a probability distribution of the second feature vector; and
a probability distribution estimation process of estimating a probability distribution of the estimated feature vector in a case where an individual falls within the second age range from the first feature vector of the individual in the first age range using the data mapping process in which the mapping parameter is updated by the update process.

The state transition estimation device may further include a memory. The memory may store a program for causing the at least one processor to execute each of the processing.

While the present disclosure has been particularly shown and described with reference to example embodiments thereof, the present disclosure is not limited to these example embodiments. It will be understood by those of ordinary skill in the art that various changes in form and details may be made therein without departing from the spirit and scope of the present disclosure as defined by the claims. And each embodiment can be appropriately combined with at least one of embodiments. Each of the drawings or figures is merely an example to illustrate one or more example embodiments. Each figure may not be associated with only one particular example embodiment, but may be associated with one or more other example embodiments. As those of ordinary skill in the art will understand, various features or steps described with reference to any one of the figures can be combined with features or steps illustrated in one or more other figures, for example to produce example embodiments that are not explicitly illustrated or described. Not all of the features or steps illustrated in any one of the figures to describe an example embodiment are necessarily essential, and some features or steps may be omitted. The order of the steps described in any of the figures may be changed as appropriate.

What is claimed is:

1. A state transition estimation device comprising:
at least one memory storing instructions; and
at least one processor configured to execute the instructions to perform:
a data input process of acquiring a first feature vector indicating a state of each of a plurality of first individuals in a first age range, a second feature vector indicating a state of each of a plurality of second individuals in a second age range older than the first age range, and individual data different from the first feature vector of each of the plurality of first individuals;
an individual variation generation process of generating a latent vector representing a variation between the first individuals from the individual data of the first individual;
a data mapping process of performing mapping from the first feature vector of the first individual to an estimated feature vector in a case where the first individual falls within the second age range using the latent vector of the first individual;
an update process of updating a mapping parameter applied to the data mapping process based on the first feature vectors and the estimated feature vectors of the plurality of first individuals and the second feature vectors of the plurality of second individuals in such a way that the mapping in the data mapping process approaches an optimal transport from a probability distribution of the first feature vector to a probability distribution of the second feature vector; and
a probability distribution estimation process of estimating a probability distribution of the estimated feature vector in a case where an individual falls within the second age range from the first feature vector of the individual in the first age range using the data mapping process in which the mapping parameter is updated by the update process.

2. The state transition estimation device according to claim 1, wherein the data mapping process includes a process of outputting an average and a variance of the estimated feature vector obtained by the mapping using a machine learning model of a variational auto encoder (VAE) type.

3. The state transition estimation device according to claim 2, wherein the probability distribution estimation process includes a process of estimating a probability distribution of the estimated feature vector in a case where an individual falls within the second age range from the individual data of the individual in the first age range and the first feature vector using a machine learning model having the individual data as an input and a difference vector between the estimated feature vector obtained by the data mapping process and an average of the estimated feature vector as an output.

4. The state transition estimation device according to claim 3, wherein
the probability distribution estimation process includes a process of estimating a first probability distribution of the estimated feature vector from the first feature vector of an individual in the first age range and estimating a second probability distribution of the estimated feature vector from the individual data of an individual in the first age range and the first feature vector, and
the processor is configured to execute the instruction to perform display processing of displaying the first probability distribution and the second probability distribution.

5. The state transition estimation device according to claim 1, wherein
at least some of the first individuals and at least some of the second individuals are relevant to each other, and
the update process updates the mapping parameter applied to the data mapping process in such a way that the estimated feature vector mapped from the first feature vectors of the at least some of the first individuals and the second feature vector of the at least some of the second individuals approach each other in the data mapping process.

6. The state transition estimation device according to claim 1, wherein
the individual data includes data that can change on a time axis,
the probability distribution estimation process includes a process of re-estimating a probability distribution of the estimated feature vector in response to a change in the individual data, and
the processor is configured to execute the instruction to perform display processing of displaying a change in a probability distribution of the estimated feature vector estimated in the probability distribution estimation process.

7. The state transition estimation device according to claim 1, wherein
the first individual, the second individual, and the individual are persons, and
the first feature vector, the second feature vector, and the estimated feature vector indicate a state related to health or a disease risk.

8. The state transition estimation device according to claim 1, wherein the processor is configured to execute the instruction to perform display processing of displaying the probability distribution estimated in the probability distribution estimation process as information for decision making on a life plan.

9. A state transition estimation method comprising:
a data input process of acquiring a first feature vector indicating a state of each of a plurality of first individuals in a first age range, a second feature vector indicating a state of each of a plurality of second individuals in a second age range older than the first age range, and individual data different from the first feature vector of each of the plurality of first individuals;
an individual variation generation process of generating a latent vector representing a variation between the first individuals from the individual data of the first individual;
a data mapping process of performing mapping from the first feature vector of the first individual to an estimated feature vector in a case where the first individual falls within the second age range using the latent vector of the first individual;
an update process of updating a mapping parameter applied to the data mapping process based on the first feature vectors and the estimated feature vectors of the plurality of first individuals and the second feature vectors of the plurality of second individuals in such a way that the mapping in the data mapping process approaches an optimal transport from a probability distribution of the first feature vector to a probability distribution of the second feature vector; and
a probability distribution estimation process of estimating a probability distribution of the estimated feature vector in a case where an individual falls within the second age range from the first feature vector of the individual in the first age range using the data mapping process in which the mapping parameter is updated by the update process.

10. A non-transitory computer readable medium having stored therein a state transition estimation program causing a computer to execute:
a data input process of acquiring a first feature vector indicating a state of each of a plurality of first individuals in a first age range, a second feature vector indicating a state of each of a plurality of second individuals in a second age range older than the first age range, and individual data different from the first feature vector of each of the plurality of first individuals;
an individual variation generation process of generating a latent vector representing a variation between the first individuals from the individual data of the first individual;
a data mapping process of performing mapping from the first feature vector of the first individual to an estimated feature vector in a case where the first individual falls within the second age range using the latent vector of the first individual;
an update process of updating a mapping parameter applied to the data mapping process based on the first feature vectors and the estimated feature vectors of the plurality of first individuals and the second feature vectors of the plurality of second individuals in such a way that the mapping in the data mapping process approaches an optimal transport from a probability distribution of the first feature vector to a probability distribution of the second feature vector; and
a probability distribution estimation process of estimating a probability distribution of the estimated feature vector in a case where an individual falls within the second age range from the first feature vector of the individual in the first age range using the data mapping process in which the mapping parameter is updated by the update process.
